SEQUENCE LISTING (12 sequences)

SEQ ID NO: 1 - DNA, 90 bp, Artificial Sequence
  Synthetic polynucleotide
  misc_feature (1)..(1): modified by /5Biosg/
gtgttcccacccraggtcgctgtgtttgagccatcagaagcagagatctcccacacccaaaaggccacactggtgtgcctggccacaggc

SEQ ID NO: 2 - DNA, 90 bp, Artificial Sequence
  Synthetic polynucleotide
  misc_feature (1)..(1): modified by /5Biosg/
ctgtctgcctattcaccgattttgattctcaaacaaatgtgtcacaaagtaaggattctgatgtgtatatcacagacaaaactgtgctag

SEQ ID NO: 3 - DNA, 90 bp, Artificial Sequence
  Synthetic polynucleotide
  misc_feature (1)..(1): modified by /5Biosg/
gacttgacagcggaagtggttgcgggggttctgccagaaggtggccgagaccctcaggcggctgctcaggcagtatctggagtcattgag

SEQ ID NO: 4 - DNA, 90 bp, Artificial Sequence
  Synthetic polynucleotide
  misc_feature (1)..(1): modified by /5Biosg/
aggctgtcttacaatcttgcagatctcagctggaccacagccgcagcgtcatgagcagattaaacccggccactttcaggaggaggattc

SEQ ID NO: 5 - DNA, 74 bp, Artificial Sequence
  Synthetic polynucleotide
aatgatacggcgaccaccgagatctacacctctctatgcctgtccgcggaagcagtggtatcaacgcagagtac

SEQ ID NO: 6 - DNA, 47 bp, Artificial Sequence
  Synthetic polynucleotide
caagcagaagacggcatacgagattcgccttagtctcgtgggctcgg

SEQ ID NO: 7 - DNA, 37 bp, Artificial Sequence
  Synthetic polynucleotide
gcctgtccgcggaagcagtggtatcaacgcagagtac

SEQ ID NO: 8 - DNA, 30 bp, Artificial Sequence
  Synthetic polynucleotide
  misc_feature (20)..(20): n is a, c, g, or t
ggtacacggcagggtcaggnttctggatat

SEQ ID NO: 9 - DNA, 42 bp, Artificial Sequence
  Synthetic polynucleotide
caaacacagcgacctcgggtgggaacacsttkttcaggtcct

SEQ ID NO: 10 - DNA, 34 bp, Artificial Sequence
  Synthetic polynucleotide
aactggtacacagcaggttctgggttctggatgt

SEQ ID NO: 11 - DNA, 36 bp, Artificial Sequence
  Synthetic polynucleotide
aggagaccttgggtggagtcacatttctcagatcct

SEQ ID NO: 12 - DNA, 23 bp, Artificial Sequence
  Synthetic polynucleotide
aagcagtggtatcaacgcagagt

(19) United States
(12) Patent Application Publication
(10) Pub. No.: US 2020/0095625 A1
(43) Pub. Date: Mar. 26, 2020

(54) METHODS FOR ANALYZING T CELL RECEPTORS AND B CELL RECEPTORS

(71) Applicant: Massachusetts Institute of Technology, Cambridge, MA (US)

(72) Inventors: Todd Michael Gierahn, Brookline, MA (US); Ang Tu, Cambridge, MA (US); J. Christopher Love, Somerville, MA (US)

(73) Assignee: Massachusetts Institute of Technology, Cambridge, MA (US)

(21) Appl. No.: 16/081,083

(22) PCT Filed: Jan. 12, 2018
(86) PCT No.: PCT/US2018/013443

§ 371 Date: Aug. 30, 2018

(51) Int. Cl. C12Q 1/6809; C12Q 1/6881; C40B 30/04; C40B 20/04; C12P 19/34

(52) U.S. Cl. CPC C12Q 1/6809; C12Q 1/6881; C40B 30/04; C12Y 207/07; C12P 19/34; C12Q 2535/122; C12Q 2565/514; C40B 20/04

(57) ABSTRACT

The invention includes methods for enriching for T cell receptor- or B cell receptor-encoding transcripts from a single cell RNA sequencing library, (ii) sequencing T cell receptor- or B cell receptor-encoding transcripts from a single cell RNA sequencing library, and (iii) targeted tagmentation of T cell receptor- or B cell receptor-encoding transcripts.

### RELATED APPLICATION

This application claims the benefit of the filing date of U.S. Provisional Application No. 62/445,350, filed on Jan. 12, 2017, the content of which is herein incorporated by reference in its entirety.

### FEDERALLY SPONSORED RESEARCH

This invention was made with Government support under Grant No. DP3 DK097681 awarded by the National Institutes of Health, and Grant No. W911NF-13-D-0001 awarded by the Army Research Office. The Government has certain rights in the invention.

### BACKGROUND OF INVENTION

T cells and B cells of the adaptive immune system recognize foreign entities by binding unknown peptides and sugar molecules with their immune receptors (T cell receptor (TCR) or B cell receptor (BCR), respectively). To enable recognition of the wide array of peptides and sugars expressed by foreign pathogens, each naïve T cell or B cell generates a unique receptor sequence through genetic recombination. The sequence of each parental cell's TCR/BCR acts as a genetic barcode that marks all subsequent progeny. Defining the sequences of these cells is important to understanding disease progression, responses to interventions such as vaccines, and developing new therapies such as immunotherapies. Approaches to characterizing these receptors from cells are therefore essential.

### SUMMARY OF INVENTION

This disclosure provides methods for obtaining, enriching, and optionally sequencing transcripts from a variety of cell types and cell mixtures, provided that some minimal length of specific and shared (or common sequence) is known. As an example, the methods may be used to enrich for sequences from a population of microbes, which share common sequence, for the purpose of identifying genetic variants amongst those microbes. A specific example may be analysis of bacterial (e.g., MRSA) or viral (e.g., Zika) microbes to understand the genetic basis for differences in potency, virulence, immunogenicity, drug-resistance, etc.

As another specific example, this disclosure provides methods suitable for analysis of T cell receptor (TCR) and/or B cell receptor (BCR) transcripts and thus ultimately TCR or BCR make up (e.g., alpha and beta chain usage in a TCR of each cell of a given cell population). Thus, provided herein are individual methods for (i) enriching for T cell receptor- or B cell receptor-encoding transcripts, e.g., from a single cell RNA sequencing library, and/or (ii) identifying a T cell receptor- or B cell receptor in a cell, e.g., by sequencing T cell receptor- or B cell receptor-encoding transcripts, e.g., from a single cell RNA sequencing library, and/or (iii) producing a library of T cell receptor- or B cell receptor-encoding transcripts, each comprising a same primer sequence at a uniform position in the variable region. The methods allow for (i) identifying the T cell receptor or B cell receptor expressed in a specific cell; (ii) quantifying the number of cells expressing different T cell receptors or B cell receptors; (iii) correlating specific T cell receptors or B cell receptors with a full transcriptional profile, cell function, and phenotype; and (iv) robust T cell receptor and B cell receptor profiling with low cell input requirements.

In one aspect, the invention provides methods for enriching for T cell receptor- or B cell receptor-encoding transcripts in a library of transcripts. The methods comprise:

providing a library of transcripts from a plurality of cells, with transcripts from each cell identified by a unique barcode (i.e., a single unique barcode is present in all the transcripts obtained from the same single cell);

contacting the library of transcripts with a labeled oligonucleotide that is complementary to a constant region of the T cell receptor- or B cell receptor-encoding transcripts; and

separating the labeled oligonucleotide hybridized to the T cell receptor- or B cell receptor-encoding transcripts from the library of transcripts,

thereby obtaining a population of transcripts enriched for T cell receptor- or B cell receptor-encoding transcripts.

In some embodiments, the methods further comprise amplifying the population of transcripts enriched for T cell receptor- or B cell receptor-encoding transcripts.

In some embodiments, the methods further comprise enriching the amplified population of transcripts enriched for T cell receptor- or B cell receptor-encoding transcripts using the methods described herein.

In another aspect, the invention provides methods for identifying a T cell receptor- or B cell receptor in a cell. The methods comprise:

providing a library of transcripts from a plurality of cells, with each transcript having a 5′ universal primer site followed by a barcode that is unique to each cell (i.e., all the transcripts harvested from a single cell are modified to have the same barcode);

contacting the library of transcripts with a first sequencing primer that is complementary to the universal primer site, and which is typically located at or near and end of the transcript including the 5′ end of the transcript, and a second sequencing primer that is complementary to a constant region of T cell receptor- or B cell receptor-encoding transcripts, and which is typically located at an internal region of the transcript;

sequencing from both primers in the same read, such that barcode sequence data for a T cell receptor- or B cell receptor-encoding transcript is generated in conjunction with CDR3 sequence data.

In yet another aspect, the invention provides methods for producing a library of T cell receptor- or B cell receptor-encoding transcripts, each comprising a same primer sequence at a uniform position in the variable region. The methods comprise providing:

(i) a library of T cell receptor- or B cell receptor-encoding transcripts from a plurality of cells;

(ii) a pool of oligonucleotides complementary to all known sequences at a uniform position in the variable region;

(iii) transposase; and

(iv) a transposon comprising the primer sequence.

In yet another aspect, the invention provides methods for determining an expression profile associated with a T cell or B cell receptor. The methods comprise:

providing a library of transcripts from a plurality of cells, with transcripts from each cell identified by a unique barcode;

determining sequence and expression level of one or more transcripts in the library;

selecting one or more sequences of a T cell receptor or B cell receptor from the library or derived from the library; and

identifying expression levels of transcripts having the same barcode as the T cell or B cell receptor;

thereby providing a gene expression profile for the T cell or B cell receptor.

### BRIEF DESCRIPTION OF DRAWINGS

FIG. 1 is a schematic diagram of a TCR/BCR Seq-Well transcript.

FIG. 2 is a schematic diagram of enriching TCR/BCR transcripts with biotinylated oligonucleotide probes. TCR transcripts are pulled down using biotinylated probes to the constant region.

FIG. 3 is a series of graphs depicting the increase in TCR/BCR transcript following three rounds of enrichment with biotinylated probes. In the left most bar graph, each set of four bars (of which there are 3 sets) correspond from left to right as follows: library, enrichment round 1, enrichment round 2, and enrichment round 3. The three sets correspond from left to right as follows: TCRA, TCRB and BCRH. Of the remaining bar graphs (of which there are four), the two top graphs and the bottom left graph each have 2 sets of three bars, and each set of three bars correspond to enrichment round 1, enrichment round 2, enrichment round 3, from left to right. The three sets correspond from left to right as follows: TCRA, TCRB and BCRH. In the last bar graph on the bottom right, each set of two bars (of which there are three) correspond to enrichment round 1 and enrichment round 2, from left to right. The three sets correspond from left to right as follows: TCRA, TCRB and BCRH.

FIG. 4 is a graph showing the fraction of reads mapped to the T cell receptor after a single round of enrichment in purified T cells, PBMC/splenocytes, and tumor cells. The purified T cells samples are represented by the 4 topmost circles in the human samples and the 3 topmost circles in the mouse samples. The only tumor sample shown is the lowest ranking circle in the mouse samples.

FIG. 5 is a schematic diagram showing sequencing primers for sequencing (i) the bead barcode region and (ii) the CDR3 and V/J region of TCR/BCR transcripts.

FIG. 6 is a schematic diagram showing sequencing primers for sequencing (i) the bead barcode region and (ii) the CDR3 and V/J region of TCR/BCR transcripts in read 1 and the transposon primer in read 2.

FIG. 7 is a series of graphs. The graph on the left is a graph of reads mapped to the T cell receptor loci. The graph on the right is a graph of reads to assembled CDR3. There are fewer mapped index reads due to tagmentation in constant region. The index reads still yielded 2× more assembled CDR3 sequences.

FIG. 8 is a series of graphs. In the top panel, the number of times a unique CDR3 sequence is found associated with a given number of bead barcodes is displayed. In the bottom left panel, the ratio of the number of cells found to express the OT-I TCRA to the number of unique T cells (1 cell/CDR3) for the three independent libraries is displayed. In the bottom right panel, the ratio of the number of cells found to express the OT-I TCRB to the number of unique T cells (1 cell/CDR3) for the three independent libraries is displayed.

FIG. 9 is a series of graphs. In the left panel, SeqWell-derived single cell transcriptomes created from leukocytes isolated from a mouse lymph node were sequenced and t-distributed stochastic neighbor embedding (rtSNE) analysis is shown. In the right panel, expression levels of TCRA (TRAC) and CD3D were then mapped onto the tSNE plot. The dots in each of the plots are color coded and a color rendering, as submitted in U.S. Provisional Application No. 62/445,350, is available in the USPTO/PAIR file history of such application.

### DETAILED DESCRIPTION OF INVENTION

### Definitions

“Oligonucleotides”, in the context of the invention, refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). Oligonucleotides include DNA such as D-form DNA and L-form DNA and RNA, as well as various modifications thereof. Modifications include base modifications, sugar modifications, and backbone modifications. Non-limiting examples of these are provided below.

Non-limiting examples of DNA variants that may be used in the invention are L-DNA (the backbone enantiomer of DNA, known in the literature), peptide nucleic acids (PNA) bisPNA clamp, a pseudocomplementary PNA, a locked nucleic acid (LNA), or co-nucleic acids of the above such as DNA-LNA co-nucleic acids. It is to be understood that the oligonucleotides used in products and methods of the invention may be homogeneous or heterogeneous in nature. As an example, they may be completely DNA in nature or they may be comprised of DNA and non-DNA (e.g., LNA) monomers or sequences. Thus, any combination of nucleic acid elements may be used. The oligonucleotide modification may render the oligonucleotide more stable and/or less susceptible to degradation under certain conditions. For example, in some instances, the oligonucleotides are nuclease-resistant.

The oligonucleotides may have a homogenous backbone (e.g., entirely phosphodiester or entirely phosphorothioate) or a heterogeneous (or chimeric) backbone. Phosphorothioate backbone modifications render an oligonucleotide less susceptible to nucleases and thus more stable (as compared to a native phosphodiester backbone nucleic acid) under certain conditions. Other linkages that may provide more stability to an oligonucleotide include without limitation phosphorodithioate linkages, methylphosphonate linkages, methylphosphorothioate linkages, boranophosphonate linkages, peptide linkages, alkyl linkages, dephospho type linkages, and the like. Thus, in some instances, the oligonucleotides have non-naturally occurring backbones.

Oligonucleotides may be synthesized in vitro. Methods for synthesizing nucleic acids, including automated nucleic acid synthesis, are also known in the art. Oligonucleotides having modified backbones, such as backbones comprising phosphorothioate linkages, and including those comprising chimeric modified backbones may be synthesized using automated techniques employing either phosphoramidate or H phosphonate chemistries. (F. E. Eckstein, “Oligonucleotides and Analogues—A Practical Approach” IRL Press, Oxford, U K, 1991, and M. D. Matteucci and M. H. Caruthers, Tetrahedron Lett. 21, 719 (1980)) Aryl and alkyl phosphonate linkages can be made, e.g., as described in U.S. Pat. No. 4,469,863; and alkylphosphotriester linkages (in which the charged oxygen moiety is alkylated), e.g., as described in U.S. Pat. No. 5,023,243 and European Patent No. 092,574, can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165; Crooke S T et al. (1996) Annu Rev Pharmacol Toxicol 36:107-129; and Hunziker J et al. (1995) Mod Synth Methods 7:331-417.

The oligonucleotides may additionally or alternatively comprise modifications in their sugars. For example, a β-ribose unit or a β-D-2′-deoxyribose unit can be replaced by a modified sugar unit, wherein the modified sugar unit is for example selected from β D-ribose, α-D-2′-deoxyribose, L-2′-deoxyribose, 2′-F-2′-deoxyribose, arabinose, 2′-F-arabinose, 2′-O—(C1-C6)alkyl-ribose, preferably 2′-O—(C1-C6)alkyl-ribose is 2′-O-methylribose, 2′-O—(C2 C6)alkenyl-ribose, 2′-[O—(C1-C6)alkyl-O—(C1-C6)alkyl]-ribose, 2′-NH2-2′-deoxyribose, β D xylo-furanose, α arabinofuranose, 2,4 dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

The oligonucleotides may comprise modifications in their bases. Modified bases include modified cytosines (such as 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil), modified guanines such as 7 deazaguanine, 7 deaza 7 substituted guanine (such as 7 deaza 7 (C2 C6)alkynylguanine), 7 deaza 8 substituted guanine, hypoxanthine, N2-substituted guanines (e.g. N2-methyl-guanine), 5-amino-3-methyl-3H,6H-thiazolo[4,5-d]pyrimidine-2,7-dione, 2,6 diaminopurine, 2 aminopurine, purine, indole, adenine, substituted adenines (e.g. N6-methyl-adenine, 8-oxo-adenine) 8 substituted guanine (e.g. 8 hydroxyguanine and 8 bromoguanine), and 6 thioguanine. The nucleic acids may comprise universal bases (e.g. 3-nitropyrrole, P-base, 4-methyl-indole, 5-nitro-indole, and K-base) and/or aromatic ring systems (e.g. fluorobenzene, difluorobenzene, benzimidazole or dichloro-benzimidazole, 1-methyl-1H-[1,2,4]triazole-3-carboxylic acid amide). A particular base pair that may be incorporated into the oligonucleotides of the invention is a dZ and dP non-standard nucleobase pair reported by Yang et al. NAR, 2006, 34(21):6095-6101. dZ, the pyrimidine analog, is 6-amino-5-nitro-3-(1′-β-D-2′-deoxyribofuranosyl)-2(1H)-pyridone, and its Watson-Crick complement dP, the purine analog, is 2-amino-8-(1′-β-D-1′-deoxyribofuranosyl)-imidazo[1,2-a]-1,3,5-triazin-4(8H)-one.

“Probes” and “Primers”, as described herein, comprise oligonucleotides. They can be nucleic acids in whole or in part. They may comprise naturally occurring nucleotides and/or non-naturally occurring nucleotides. They may be or may comprise DNA, RNA, DNA analogs, RNA analogs, PNA, LNA and combinations thereof, provided it is able to hybridize in a sequence-specific manner to oligonucleotides and/or to be conjugated in some instances to a label.

The probe or primer may form at least a Watson-Crick bond with the target. In other instances, the probe or primer such as the probe may form a Hoogsteen bond with the target, thereby forming a triplex. A probe or primer that binds by Hoogsteen binding enters the major groove of a nucleic acid and hybridizes with the bases located there. In some embodiments, the probes or primers can form both Watson-Crick and Hoogsteen bonds with the target. BisPNA probes, for instance, are capable of both Watson-Crick and Hoogsteen binding to a nucleic acid.

The probe or primer can be any length including but not limited to 8-100 nucleotides, 8-75 nucleotides, 8-50 nucleotides, 8-30 nucleotides, 18-30 nucleotides, and every integer therebetween as if explicitly recited herein.

The probes or primers are preferably single stranded, but they are not so limited. For example, when the probe or primer is a bisPNA it can adopt a secondary structure with the target resulting in a triple helix conformation, with one region of the bisPNA forming Hoogsteen bonds with the backbone of the identifier sequence and another region of the bisPNA forming Watson-Crick bonds with the bases of the target.

Hybridization: The binding of the probe or primer to the target via hybridization can be manipulated based on the hybridization conditions. For example, salt concentration and temperature can be modulated. Those of ordinary skill in the art will be able to determine optimum conditions for a desired specificity. In some embodiments, the hybridization conditions are stringent so that only completely complementary probes or primers will bind to the target. In other embodiments, less than stringent conditions are used.

Sequence-dependent binding when used in the context of a nucleic acid hybridization means recognition and binding to a particular linear arrangement of nucleotides in the nucleic acid. In the case of probes and primers, the linear arrangement includes contiguous nucleotides that each binds to a corresponding complementary nucleotide in the probes and primers.

The probes and primers described herein hybridize to their target nucleic acids, typically under stringent conditions. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH7), 0.5% SDS, 2 mM EDTA). SSC is 0.15M sodium chloride/0.015M sodium citrate, pH 7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit specific and selective hybridization of probes and/or primers to the nucleic acids of the invention (e.g., by using lower stringency conditions).

Each T cell lineage and B cell lineage has a unique T cell receptor and B cell receptor, respectively. Since the specificity and parental lineage of each T/B cell is encoded in their TCR/BCR, it is useful to efficiently sequence these receptors in clinical samples both for monitoring responses to vaccines or immunotherapy interventions and utilizing the sequences themselves to generate patient specific immunotherapies in the form of adoptive T cell therapy or recombinant antibodies. Moreover, identifying the unique T cell receptors and B cell receptors in individual cells will allow for the observation and characterization of interactions between immune cells.

TCR/BCR transcripts can be sequenced from a bulk population cells or from single cells. The former can yield a relatively large number of TCR/BCR sequences but no information about which or how many cells are expressing a given receptor sequence. Alternatively, in single cell RNA sequencing, the T cell receptor or B cell receptor sequences of a specific cells can be identified.

Provided herein are methods for (i) enriching for T cell receptor- or B cell receptor-encoding transcripts, e.g., from a single cell RNA sequencing library, (ii) identifying a T cell receptor- or B cell receptor in a cell, e.g., by sequencing T cell receptor- or B cell receptor-encoding transcripts, e.g., from a single cell RNA sequencing library, and (iii) producing a library of T cell receptor- or B cell receptor-encoding transcripts, each comprising a same primer sequence at a uniform position in the variable region. The methods allow for (i) identifying the T cell receptor or B cell receptor expressed in a specific cell; (ii) quantifying the number of cells expressing different T cell receptors or B cell receptors; (iii) correlating specific T cell receptors or B cell receptors with a full transcriptional profile, cell function, and phenotype; (iv) robust T cell receptor and B cell receptor profiling with low cell input requirements.

### T Cell Receptors

T-cell receptors are expressed in nature on the surface of T-cells usually as alpha/beta and gamma/delta heterodimeric integral membrane proteins, each subunit comprising a short intracellular segment, a single transmembrane alpha-helix and two globular extracellular Ig-superfamily domains. The TCR-heterodimer is stabilized by an extracellular, membrane proximal, inter-chain disulphide bond (Immunobiology. 5th ed. Janeway, Charles A.; Travers, Paul; Walport, Mark; Shlomchik, Mark. New York and London: Garland Publishing; 2001). TCRs therefore have four extracellular domains, the two membrane proximal (C-terminal) domains, which are constant, and the two N-terminal domains, which are variable. The variable region are encoded by variable gene segments which are rearranged with junctional and constant gene segments (and diversity gene segments in the case of (3-chains) to produce the TCR diversity observed in the mature immune system.

The variable regions of both TCR chains contain three hypervariable loops, or complementarity-determining regions (CDRs). CDR3 is closest to the constant region.

### B Cell Receptors

The B-cell receptor or BCR is a transmembrane receptor protein located on the outer surface of B cells. The receptor's binding moiety is composed of a membrane-bound antibody.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. An antibody can have the structural features of IgA, IgG, IgE, IgD, IgM (as well as subtypes thereof).

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3.

### Single Cell Libraries

The methods provided herein utilize libraries. In some embodiments, the libraries are single cell libraries. In some embodies, the libraries are generated by single cell RNA sequencing. Methods for single cell RNA sequencing include, but are not limited to, DropSeq, InDrop, 10× Genomics, and SeqWell.

In SeqWell, an array of >80,000 sub-picoliter wells are used to isolate single cells and a barcoded transcript capture bead. A semi-porous membrane is used to seal the wells, preventing escape of macromolecules, such as mRNA, while allowing passage of small molecule lysis buffers. This enables robust cell lysis within the sealed compartments and capture of the mRNA molecules on the beads. After capture, a barcode that is unique to each bead (and therefore, each well) is fused to each transcript captured in a well during reverse transcription. The barcoded cDNA libraries undergo whole transcriptome amplification (WTA) and are sequenced. Single cell transcriptomes are recovered in silico by aggregating all the transcripts with the same bead barcode. SeqWell enables acquisition of 1000s of TCR/BCR sequences that can be attributed to specific cells through a DNA barcode, enabling counting the number of cells expressing a given BCR/TCR as well as correlating TCR/BCR usage to the transcriptional profile of each cell. Furthermore, the technique is optimized for an input cell number of 10,000 cells or less, making it ideal for extracting this information from cells present in clinical tissue samples. In contrast to SeqWell, in which thousands of cells are analyzed at once, in conventional single cell RNAseq, single T or B cells are sorted based on a limited number of pre-defined markers.

In some embodiments, the library comprises transcripts from a plurality of cells. In some embodiments, a plurality of cells comprises 100, 1,000, 10,000, 100,000 or more cells. In some embodiments, the library is prepared using, e.g., the SeqWell, InDrop, DropSeq, or 10× Genomics methods and a plurality of cells comprises between 10,000 and 1,000,000 cells.

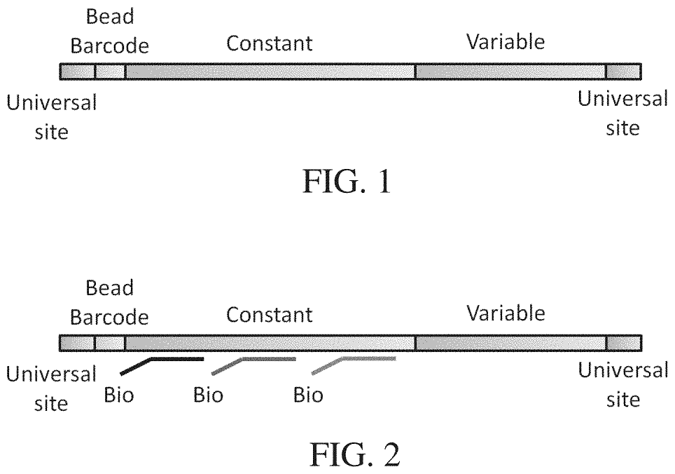

In some embodiments, the transcripts in the library have a barcode and further comprise universal primer sites at the 5′ and 3′ ends, as is shown in FIG. 1.

In some embodiments, in the library, every transcript from a single cell (i.e., the same single cell) has the same barcode and transcripts from different cells have different barcodes. In some embodiments, a barcode is 3, 4, 5, 6, 7, 8, 9, 10, 11, 12, 13, 14, 15, 16, 17, 18, 19, 20, 21, 22, 23 24, 25, 26, 27, 28, 29, or 30 nucleotides, e.g., is from 10 to 20 nucleotides long.

As used herein, a “universal primer site” is an exogenous primer binding site introduced into the nucleic acid molecule for the purpose of primer binding. Examples of universal primer sites include p5 and nexterra.

### Enrichment of T Cell Receptor/B Cell Receptor Transcripts

In any of the single cell RNA sequencing methods described herein, transcripts encoding the TCR and BCR are captured and barcoded along with the rest of the transcriptome. The low frequency of these transcripts in the library makes some form of enrichment necessary. One option for TCR/BCR enrichment comprises PCR using primers specific for the constant region and the universal primer site at the 5′ end of the transcript. Another option comprises PCR using primers specific for the universal primer site at the 5′ end of the transcript and a pool of >30 primers specific for all the possible variable regions. One shortcoming of these methods is that the PCR product will not contain the barcode sequence and, thus, it will not be possible associate the TCR/BCR sequence with a particular cell. Another shortcoming of these methods is that primers that bind to the constant region and/or variable region with high stringency are required.

In some embodiments, methods for enriching full length TCR/BCR transcripts from the library utilize a single or small panel of biotinylated 90-mer oligonucleotides complementary to the constant region of the TCR or BCR to bind the immune receptor transcripts and then pull these complexes out of solution using streptavidin beads, as is shown in FIG. 2. It is to be understood that other affinity pairs (or binding pairs) can be used instead of biotin and streptavidin, and that the use of biotin and streptavidin in this disclosure is meant to be illustrative and not limiting.

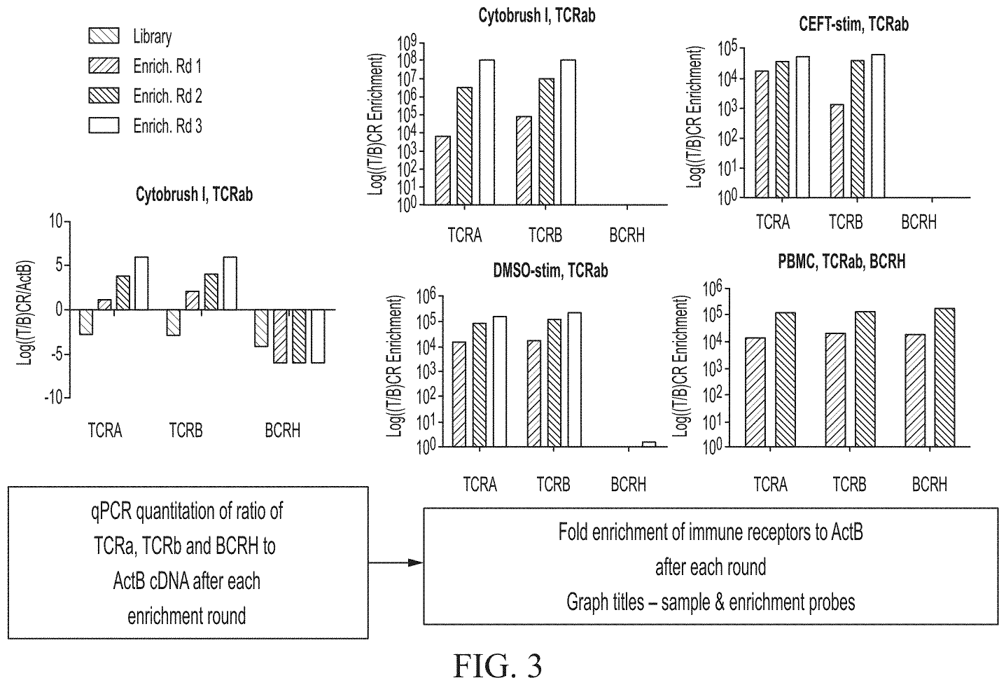

A single round of enrichment yields libraries that are >50% TCR or BCR transcripts, a fold enrichment of 10³-10⁴. As is shown in FIG. 3 and is described further in Example 2, subsequent rounds of enrichment further increase the level of TCR/BCR transcript.

Critically, this approach enriches the entire BCR/TCR cDNA molecule including the 3′ bead barcode which can be used to link the TCR/BCR sequence to the rest of the transcriptome of the cell. Also, by creating full length libraries, the entire immune receptor variable region can be sequenced. This is important for creating recombinant antibodies from the BCR sequences. Also, since the technique only requires 1-3 probes (1, 2 and/or 3 probes) specific to the constant regions of the receptors, the enrichment strategy is easily applied to any species with an annotated genome as opposed to approaches using pools of >30 primers which require identification of all the variable regions in the immune receptor locus of the species as well as QC of all the primers. Further, because 100% identity to the constant region is not required, the methods described herein can be used to enrich TCR/BCR transcripts from various species in which the sequence of the constant region is not known.

In some embodiments, the methods for enrichment comprise providing a library of transcripts from a plurality of cells, with transcripts from each cell identified by a unique barcode; contacting the library of transcripts with a labeled oligonucleotide that is complementary to the constant region of the T cell receptor- or B cell receptor-encoding transcripts; and separating the labeled oligonucleotide hybridized to the T cell receptor- or B cell receptor-encoding transcripts from the library of transcripts.

In some embodiments, the oligonucleotide is a probe.

In some embodiments, the labeled oligonucleotide hybridized to the T cell receptor- or B cell receptor-encoding transcripts is separated from the library by binding of the labeled oligonucleotide to a binding partner. In some embodiments, the binding partner is conjugated to a bead. The labeled oligonucleotide or probe typically intends that the oligonucleotide or probe is conjugated, covalently or non-covalently, to one member of an affinity pair or of a binding pair. The members of a binding pair may be referred to as binding partners.

The binding partners may include without limitation antibodies including but not limited to single chain antibodies, antigen-binding antibody fragments, antigens (to be used to bind to their antibodies, for example), receptors, ligands, aptamers, aptamer receptors, small molecules, and the like, provided they are a member of a binding pair, with the understanding that the other member of the binding pair is present on the bead used for extraction or physical separation. Examples of binding pairs include biotin and avidin or streptavidin, antibody (or antibody fragment) and antigen, receptor and receptor ligand, aptamer and aptamer ligand, and the like.

The linkage between the oligonucleotide and the binding partner may be covalent or non-covalent depending on the strength of binding required for a particular application. Labeled oligonucleotides may be purchased commercially or they may be synthesized, for example, by first incorporating a reactive group (or moiety) into the oligonucleotide, including at or near one of its ends, and then reacting this group (or moiety) with the binding partner of interest which may or may not be modified itself. Suitable reactive groups are known in the art. Examples of reactive groups that can covalently conjugate to other reactive groups (leading to an irreversible conjugation) include but are not limited to amine groups (which react to, for example, esters to produce amides), carboxylic acids, amides, carbonyls (such as aldehydes, ketones, acyl chlorides, carboxylic acids, esters and amides) and alcohols. Those of ordinary skill in the art will be familiar with other “covalent” reactive groups. Virtually any reactive group may be used, provided it participates in an interaction of sufficient affinity to prevent dissociation of the binding partner from its oligonucleotide.

In some embodiments, the oligonucleotide is labeled with biotin and the affinity binding partner is streptavidin. In some embodiments, the oligonucleotide is labeled with streptavidin and the affinity binding partner is biotin.

In some embodiments, the oligonucleotide is 30, 40, 50, 60, 70, 80, 90, 100, 110, or 120 nucleotides, e.g., 90 nucleotides.

In some embodiments, the oligonucleotide has less than 100% identity to the constant region sequence. In some embodiments, the oligonucleotide has 95%, 90%, 85%, or 80% or less identity to the constant region sequence.

In some embodiments, the enriched TCR/BCR sequences are then amplified using the original WTA primers, which are complementary to the 5′ and 3′ universal primer sites. Once the enriched TCR/BCR sequences are amplified, the enrichment can be repeated, e.g., can be repeated 1, 2, 3 or more times.

### T Cell Receptor/B Cell Receptor Transcript Sequencing

One approach for sequencing transcripts generated by single cell RNA sequencing includes producing random tagmented libraries. In random tagmented libraries, transposase can be used to randomly insert transposons into the transcript. The transcript can then be sequenced via paired end sequencing using a primer that is complementary to the universal primer site upstream from the barcode sequence and a primer that is complementary to the transposon sequence.

This method has disadvantages in the context of T cell receptor and B cell receptor sequence analysis. As is described above, the T cell receptor and the B cell receptor comprise variable and constant regions, with the variable region being at the 5′ end of the molecule. Because of the length of the transcript, which is usually greater than 1500 base pairs, most of the tagmented library nucleic acid molecules do not include the CDR3 region/any of the variable region, which is required to determine which T cell receptor or B cell receptor is present. One approach to increase the length of the tagmented library nucleic acid molecules is to gel purify the nucleic acid molecules and size select nucleic acid molecules. One disadvantage of this approach is that only a small fraction of nucleic acid molecules contain the CDR3 region/any of the variable region and selecting for such a small proportion of the library selects for mutants.

Provided herein are methods for determining the sequence of a T cell receptor or B cell receptor expressed in an individual cell. The methods comprise sequencing the T cell receptor or B cell receptor transcript from both the universal primer site upstream of the barcode and the constant region just upstream of the variable region in the same sequencing reaction.

The methods described herein are advantageous for at least the following reasons. The methods allow for the identification of barcode sequence information associated with the variable region sequence information, allowing the identification of specific T cell receptors or B cell receptors in specific cells. In particular, the pairing of a specific alpha chain with a specific beta chain can be determined. Furthermore, the specific alpha-beta pairing can be linked to a functional outcome.

In some embodiments, the methods comprise providing a library of transcripts from a plurality of cells, with each transcript having a universal primer site followed by a barcode that is unique to each cell; contacting the library of transcripts with a first sequencing primer that is complementary to the universal primer site and a second sequencing primer that is complementary to the constant region of T cell receptor- or B cell receptor-encoding transcripts; and sequencing from both primers in the same read, such that barcode sequence data for a T cell receptor- or B cell receptor-encoding transcript is generated in conjunction with CDR3 sequence data.

As is used herein, “in the same read” means in the same sequencing reaction. Same reads can be isolated and/or sequenced together, typically due to physical isolation from other reads on a sequencing platform. This allows an end user to identify the transcript sequence of interest (e.g., the CDR3 sequence) as well as the barcode sequence. The end user may then identify alpha and beta chain sequences from single cells, and thus recreate the TCR of such single cells, as an example. A similar approach may be taken for B cells with respect to light and heavy antibody chains.

Shown in FIG. 5 is a sequencing primer that targets the constant region of the T cell receptor or B cell receptor just upstream of the variable region. In some embodiments, constant region primer is less than 5, 10, 20, 30, 40, or 50 residues from CDR3. In the methods described herein, this constant region specific primer can be used in conjunction with the 5′ universal primer to sequence transcripts from a single cell RNA sequencing library described herein. The constant region specific primer can provide the sequence of a portion of the variable region and the universal primer can provide the sequence of the barcode. The barcode sequence information is associated with the variable region sequence, allowing the identification of specific T cell receptors or B cell receptors in specific cells.

The type of sequencing performed can be, for example, pyrosequencing, single-molecule real-time sequencing, ion torrent sequencing, sequencing by synthesis, sequencing by ligation (SOLiD™), and chain termination sequencing (e.g., Sanger sequencing). Sequencing methods are known in the art and commercially available (see, e.g., Ronaghi et al.; Uhlén, M; Nyrén, P (1998). “A sequencing method based on real-time pyrophosphate”. Science 281 (5375): 363; and Ronaghi et al.; Karamohamed, S; Pettersson, B; Uhlén, M; Nyrén, P (1996). “Real-time DNA sequencing using detection of pyrophosphate release”. Analytical Biochemistry 242 (1): 84-9.; and services and products available from Roche (454 platform), Illumina (HiSeq and MiSeq systems), Pacific Biosciences (PACBIO RS II), Life Technologies (Ion Proton™ systems and SOLiD™ systems)).

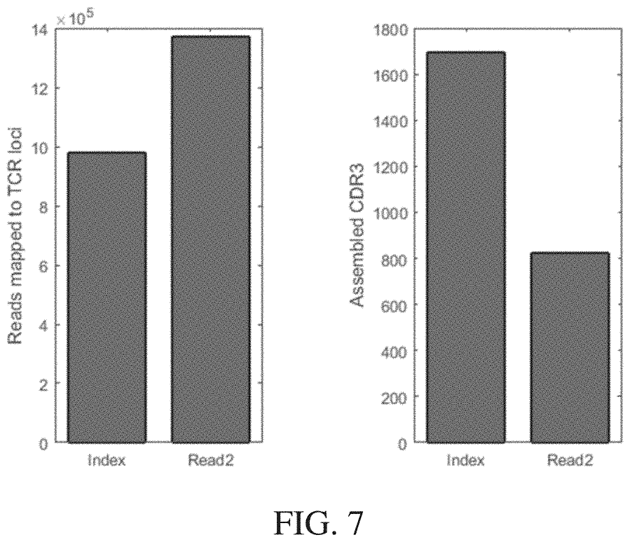

As is shown in FIG. 6, in some embodiments, the methods described herein can further comprise a second read (e.g., sequencing reaction). The methods can be performed on a tagmented library nucleic acid molecule size selected such that it will have a transposon insertion in the variable region. In the first read, the constant region specific primer can be used in conjunction with the universal primer upstream from the barcode sequence. In the second read, a primer complimentary to the transposon can be used to sequence the variable region in the opposite direction. This allows for greater sequence information for the variable region and improved sequence quality. As is shown in FIG. 7, although the transposon primer yields greater reads mapped to T cell receptor loci, the constant region specific primer yielded 2× more assembled CDR3 sequences.

### Targeted Tagmentation for Efficient Sequencing Library Creation

As is described above, the use of random tagmented libraries in the sequencing of TCR/BCR transcripts is disadvantageous. Random tagmentation can insert sequencing primer sites near the desired variable region at some frequency but the vast majority of insertions occur in undesired locations, making library creation highly inefficient. Indeed, standard protocols result in >95% of reads landing in the constant region of the transcripts, yielding no useful data.

One strategy for sequencing TCR/BCR transcripts from single cell RNA sequencing libraries is described above.

Another strategy, described herein, is targeted tagmentation. Tagmentation involves the use of transposase to insert transposons which can be used as primer sites into TCR/BCR transcripts from single cell RNA sequencing libraries.

Transposases are encoded by transposons and are well known to one skilled in the art. A transposase, as used herein, is an enzyme which catalyzes the transposition of a transposable nucleic acid sequence into the genome of a cell. Transposases and transposable DNA elements, referred to as transposons, have been discovered in almost all organisms. The genetic structures and transposition mechanisms of various transposons are summarized in the art, for example in “Transposable Genetic Elements” in “The Encyclopedia of Molecular Biology,” Kendrew and Lawrence, Eds., Blackwell Science, Ltd., Oxford (1994) and in “Mobile DNA II,” Craig, Gellert, and Lambowitz, Eds., American Society of Microbiology, Washington, D.C. (2002), which are incorporated herein by reference.

Targeting of the transposon insertion site is enabled to a desired location within the variable region. The method comprises providing: (i) a library of T cell receptor- or B cell receptor-encoding transcripts; (ii) a pool of oligonucleotides complementary to all known sequences at a uniform position in the variable region; (iii) transposase; and (iv) a transposon comprising the primer sequence. A pool of oligonucleotides complementary to all known sequences at a uniform position in the variable region comprises oligonucleotides complimentary to each known TCR or BCR at the same position in each TCR or BCR. Since transposon function requires double stranded DNA, transposon insertions will be driven to the location hybridized by the oligos.

Targeted tagmentation is particularly useful for BCR sequencing since the entire variable region must be sequenced to enable recombinant antibody production. This new approach enables the targeted insertion of transposons and hence sequencing primer sites at appropriately spaced sites to enable efficient sequencing of the entire variable region.

### Determining Gene Expression Profiles

Provided herein are methods for determining a T cell or B cell expression profile. The methods provided herein allow for the (i) enrichment and (ii) sequencing of T cell receptors and B cell receptors from single cell RNA sequencing libraries. The single cell RNA sequencing technologies described herein can be used to determine the sequence and expression level of the transcriptome. Using in silico analysis, the transcripts identified in the single cell RNA sequencing can be paired with the sequence of T cell receptors and B cell receptors from a particular cell based on barcode identity. This allows for the identification of the transcriptome associated with a particular T cell or B cell receptor.

The following Examples are included for purposes of illustration and are not intended to limit the scope of the invention.

### EXAMPLES

### Example 1: TCR/BCR Enrichment from SeqWell Library

### Reagents

Reagents needed are SeqWell WTA library; Gen® Lockdown® Reagents (IDT-1072281): 10× Wash 1, 10× Wash 2, 10× Wash 3, 10× Stringent Wash buffer, 2× Bead Wash buffer, 2× Hybridization buffer, and Hybridization Enhancer; Dynabeads M-270 Streptavidin (Thermo Fisher, Cat #6530); SeqWell WTA primer-(AAGCAGTGGTATCAACGCAGAGT) (SEQ ID NO: 12); Kapa HiFi 2× mix; Nextera XT kit; SeraPure purification beads (See Appendix 1).

### Serapure Binding Buffer

Reagents needed are 9 g PEG-8000, 10 mL 5 M NaCL, 500 μL 1 M Tris-HCL, and 100 μL 0.5 M EDTA. Add water up to 50 mL and store at 4° C. The Biotinylated TCR/BCR probes are 90-mer Biotinylated DNA Ultramers from IDT resuspended at 40 μM in water. They are: TCRBC-1-/5Biosg/GTGTTCCCACCCRAGGTCGCTGTGTTTGAGCCATCAGAAGCAGAGATCTCCCA CACCCAAAAGGCCACACTGGTGTGCCTGGCCACAGGC (SEQ ID NO: 1), TCRAC-1-/5Biosg/CTGTCTGCCTATTCACCGATTTTGATTCTCAAACAAATGTGTCACAAAGTAAG GATTCTGATGTGTATATCACAGACAAAACTGTGCTAG (SEQ ID NO: 2), TCRBC-3comp-/5Biosg/GACTTGACAGCGGAAGTGGTTGCGGGGGTTCTGCCAGAAGGTGGCCGAGACC CTCAGGCGGCTGCTCAGGCAGTATCTGGAGTCATTGAG (SEQ ID NO: 3), and TCRAC-3comp-/5Biosg/AGGCTGTCTTACAATCTTGCAGATCTCAGCTGGACCACAGCCGCAGCGTCATG AGCAGATTAAACCCGGCCACTTTCAGGAGGAGGATTC (SEQ ID NO: 4). The PCR primers are P5-TSO_Hybrid_N502-AATGATACGGCGACCACCGAGATCTACACCTCTCTATGCCTGTCCGCGGAAGCAGTG GTATCAACGCAGAGT*A*C (SEQ ID NO: 5) and Nextera_N701-CAAGCAGAAGACGGCATACGAGATTCGCCTTAGTCTCGTGGGCTCGG (SEQ ID NO: 6). The custom sequencing primers are ReadlCustomSeqB-GCCTGTCCGCGGAAGCAGTGGTATCAACGCAGAGTAC (SEQ ID NO: 7), hTCRa-GGTACACGGCAGGGTCAGGITTCTGGATAT (SEQ ID NO: 8), hTCRb-CAAACACAGCGACCTCGGGTGGGAACACSTTKTTCAGGTCCT (SEQ ID NO: 9), mTCRa-AACTGGTACACAGCAGGTTCTGGGTTCTGGATGT (SEQ ID NO: 10), and mTCRb-AGGAGACCTTGGGTGGAGTCACATTTCTCAGATCCT (SEQ ID NO: 11)

### Equipment

Equipment needed is a PCR cycler and 1.5 mL tube magnet (preferably 16 sample version).

### TCR Enrichment

Note that performing enrichments in a 96-well plate with a multi-channel pipet leads to ˜10× less enrichment. In addition, it is critical that hybridization be kept at 65° C. throughout all steps until the final three washes. The steps should be performed next to the thermal cycler and should be performed one at a time and very quickly for optimal enrichment.

All wash buffers were thawed and made in 1× solutions. 200-400 μL are needed per sample. The buffers are good for several months and may be made in large batches. Wash buffer 1 may require heating to dissolve.

Separate equi-molar mixes of TCRa and TCRb biotinylated probes were made with final total oligo concentrations of 4 μM. The TCRa/b needed to be enriched separately because TCRb transcripts are ˜10× more common and dominate sequencing if done together.

The hybridization mixes for TCRa and TCRb were made in PCR tube as follows: 8.5 μL 2× IDT Hybridization solution, 2.7 μL Hybridization Enhancer, 1 μL SeqWell WTA library (1-10 ng/μL), 0.5 μL 34 μM WTA primer (blocking oligo), 1 μL 4 μM biotinylated probe mix (Capture probes), and 3.3 μL water. The tubes were placed in the thermal cycler and run with the program: 95° C. for 10 min, 65° C. forever. The mix was incubated at 65° C. for 30 min. While the mix was incubating at 65° C., 100 μL of Wash Buffer 1 and 2 aliquots of Stringent wash buffer/enrichment were placed into 3 PCR tubes and placed in the same thermal cycler. Also during incubation, the streptavidin beads were prepared: 2 mL 1× Bead Wash buffer was made, 25 μL streptavidin beads/enrichment were added into a single 1.5 mL tube, the tube was placed on the magnet, the supernatant was removed, the product was washed 2× with 500 μL bead wash buffer, the product was resuspended in 2 μL/enrichment reaction of bead wash buffer, and the product was dispensed in 25 μL aliquots into 1.5 mL tubes, 1/enrichment.

After 30 min at 65° C., streptavidin beads were added to the enrichment by doing the following one reaction at a time: the tube containing streptavidin beads was placed on the magnet, the supernatant was removed without allowing the beads to dry, the tube was brought to the cycler, the hybridization mix tube was opened on the cycler and the mix was removed with a pipette and transferred to the tube containing streptavidin beads, and the beads were pipetted 3× and transferred quickly back to the same PCR tube in the cycler. The tube was incubated for 12 minutes and then removed from the cycler, quickly vortexed, and replaced in the cycler. The incubation and vortexing steps were repeated 2 more times. During the last incubation, 3 empty 1.5 mL/enrichment tubes were prepared. Only one tube was labelled with enrichment name.

Once the final 12 minute incubation was complete, a wash was performed with Wash 1 buffer, again one enrichment at a time: 100 μL of preheated Wash 1 buffer was transferred to hybridization mix, the entire reaction volume was transferred to one of the empty 1.5 mL tubes preloaded on the magnet, the supernatant was immediately removed, the tube was removed from the magnet, 200 μL of preheated stringent wash buffer was transferred to the tube, the mixture was pipetted up and down 3×, the volume was transferred back to the PCR tube that originally contained the stringent wash buffer on the cycler, and the 1.5 mL tube was discarded. The mixture was incubated for 5 minutes, or as long as it took to exchange all enrichment reactions.

The second stringent wash was performed, one tube at a time: the reaction was transferred to the second empty 1.5 mL tube preloaded on magnet, the supernatant was immediately removed, the tube was removed from the magnet, the second preheated aliquot of Stringent buffer was transferred to the beads, the mixture was pipetted 3×, the reaction was transferred to the PCR tube that previously contained the stringent buffer cycler, and the 1.5 mL tube was discarded. The mixture was incubated for 5 minutes.

The reactions were transferred to the labelled 1.5 mL tube preloaded on magnet and the supernatant was immediately removed. 200 μL RT wash buffer 1 were added. The tube was removed from the magnet. At this point, all tubes were processed at the same time. The tubes were vortexed on high for 2 minutes and placed on the magnet. The supernatant was removed, including any bubbles. 200 μL RT wash buffer 2 were added, and the mixture was vortexed on high for 1 minute. The tube was placed back on the magnet and the supernatant was removed. 200 μL of Wash buffer 3 were added, and the tubes were vortexed for 30 seconds. The tubes were placed back on the magnet, and the supernatant was removed and replaced with 50 μL water.

The beads were split into 2 PCR tubes. 25 μL Kapa HiFi mix and 1 μL 40 μM WTA primer was added to one of the tubes. The other tube was placed at 4° C. The enriched library was amplified as follows: 95° C. for 3 minutes, then 22 cycles of 98° C. for 20 seconds, 67° C. for 20 seconds, and 72° C. for 1 minute, then 72° C. for 1 minute and 4° C. forever.

### Purification of Enriched Library

The SeraPure beads were allowed to come to room temperature (30 minutes) and occasionally vortexed for 5-10 seconds. SeraPure beads were much preferred as they allow size selection in the 0.8-1 kb range needed for final sequencing library. 0.75× SeraPure beads were added to the pooled PCR product. Product was incubated 5 minutes. The tube was placed in the magnet stand and the beads were allowed to aggregate on the magnet (˜1-2 min). The supernatant was removed, 400 μL 80% ethanol were added to the tube, and the position of the tube on the magnet was rotated 4× to make the beads move through the volume of the tube. The wash was removed. The ethanol, magnet, and wash steps were repeated.

After removing the second wash, the top of the tube was closed, placed in centrifuge, and spun at max speed for 10 seconds. The tube was then placed back in the magnet rack and the remaining liquid was removed with a 20 μL pipet. The tube was incubated for 5 minutes with the top open to dry the pellet. The tube was removed from the magnet rack, the beads were resuspended in 13 μL water, and the tube was incubated for 2 minutes. The tube was placed on the magnet and 10 μL of supernatant were removed and placed in a new tube. For optimal enrichment, the entire procedure was repeated with 4 μL of enriched library, done on the same day. The expected TCR enrichments from a single round were based on input cell type: pure T cells yielded 75% TCR and likely did not need a second round, PBMC cells yielded 30-50% TCR, and tissue/tumor cells yielded 1-5% TCR and definitely required a second round. The library was then quantified using BioAnalyzer.

### Tagment Enriched Library

The lid of the thermal cycler was preheated. 6 μL of 150 pg/μL solution were made of enriched library, and 5 μL were transferred to a PCR tube. 10 μL of Nextera TD buffer were added, and 1.5 μL Nextera Amplicon Tagment enzyme was added to 4.5 μL tagmentase dilution buffer in a separate PCR tube. 5 μL diluted enzyme were transferred to the library solution and pipetted up and down 5 times. The tube was transferred to the thermal cycler, where it was performed: 55° C. for 5 minutes, 10° C. forever. When the solution reached 10° C., 5 μL Nextera NT solution was added and it was pipetted up and down 5×. The tube was incubated 5 minutes. Next, the following was added to the reaction: 8 μL water, 1 μL 10 μM P5-TSO_Hybrid_N5XX, 1 μL 10 μM N701, and 15 μL Nextera PCR mix. P5-TSO_Hybrid_N5XX was required if TCR constant region sequencing primer was going to be used. It required indexing to be accomplished by index 2 read. Otherwise, standard P5-TSO_Hybrid oligo could be used. The following PCR program was run: 95° C. for 30 seconds, then 16 cycles of 95° C. for 10 seconds, 55° C. for 30 seconds, 72° C. for 30 seconds, then 72° C. for 5 minutes and 4° C. forever.

### Size-Selecting Library

The SeraPure beads were allowed to come to room temperature (30 minutes) and occasionally vortexed for 5-10 seconds. SeraPure beads were much preferred as they allow size selection in the 0.8-1 kb range for final sequencing library. 0.7× SeraPure beads were added to the tagmentation reaction. Ratios varied slightly with bead batch. The reaction was incubated 5 minutes. During incubation, the SeraPure Binding buffer (SBB) was diluted with 0.7× water by mixing 500 μL SBB and 350 μL water in a separate tube. The tube was placed in the magnet stand and the beads were allowed to aggregate on the magnet (˜1-2 min). The supernatant was removed and the tube was removed from the magnet. The beads were resuspended in 400 μL SBB by vortexing and then briefly spinning to get all the solution back down. The tube was placed on the magnet for ˜3 min, until the beads pelleted. The supernatant was removed and 400 μL 80% ethanol was added to the tube. The position of the tube on the magnet was rotated 4× to make the beads move through the volume of the tube. The wash was removed. The ethanol, magnet, and washing steps were repeated.

After removing the second wash, the top of the tube was closed and it was placed in the centrifuge. The tube was spun at max speed for 10 seconds, after which it was placed back in the magnet rack. The remaining liquid was removed with a 20 μL pipet. The tube was incubated for 5 minutes with the top open to dry the pellet. Then, it was removed from the magnet rack, the beads were resuspended in 15 μL water, and the tube was incubated for 2 minutes. It was then placed back on the magnet. 12 μL of supernatant were removed and placed in a new tube. The library was quantified using BioAnalyzer. It was critical that the library be ˜900-1100 bp in size with few <700 bp. Otherwise, many reads were wasted on constant region. If the library size was too small, the SeraPure purification was repeated.

### Sequencing Library

Using TCRc sequencing primer (only tested on MiSeq to date): Read1-20 bp-primer=ReadlCustomSeqB, Index 1-150 bp-primer=mix of TCRa/TCRb sequencing primers from correct species/NO STANDARD PRIMERS, and Index 2-8 bp index-primer-none.

Using standard reverse read: Read1-20 bp-primer=ReadlCustomSeqB, Index 1-8 bp-primer-standard Nextera, and Read2-150 bp-primer-standard Nextera Read2.

### Appendix 1—SeraPure Purification Beads

SeraPure beads are 100× cheaper than Ampure XP/purification beads and provide better control over size selection. They were taken from B. Faircloth & T. Glenn, Ecol. and Evol. Biology, Univ. of California-Los Angeles. Materials are: Sera-mag SpeedBeads (Fisher #09-981-123), PEGN8000 (Amresco 0159), 0.5 M EDTA, pH 8.0 (Amresco E177), 1.0 M Tris, pH 8.0 (Amresco E199), Tween 20 (Amresco 0777), 5 M NaCl, and Fermentas ladder(s) (Ultra-low range: Fisher # FERSM1211, 50 bp: FERSM0371)

In a 50 mL conical tube using sterile stock solutions, the TE was prepared (10 mM TrisNHCl, 1 mM EDTA=500 μL 1 M Tris pH8+100 μL 0.5 M EDTA, filled conical to 50 mL mark with dH₂O). The SerNmag SpeedBeads were mixed and 1 mL was transferred to a 1.5 mL microtube. The SpeedBeads were placed on a magnet stand until the beads were drawn to the magnet. The supernatant was removed with a P200 or P1000 pipetter. The magnet and supernatant removal steps were repeated twice more. 1 mL Te was added to the beads and the tube was removed from the magnet. The mixture was fully resuspended and the microtubule was set in a rack. 9 g PEGN8000 were added to a new 50 mL, sterile conical tube. 10 mL 5 M NaCl (or 2.92 g) were added to conical), then 500 μL 1 M TrisNHCl, then 100 μL 0.5 M EDTA. The conical was filled to ˜49 mL using sterile dH₂O by eye. The conical was mixed for ˜15 minutes, until the pEG went into the solution (which became clear). 27.5 μL Tween 20 were added to conical and mixed gently. 1 mL SpeedBead+TE solution was mixed and transferred to 50 mL conical. The conical was filled to the 50 mL mark with dH₂O (if not already there) and gently mixed until brown. The solution was tested against AMPure XP using aliquots of ladder (Fermentas GeneRuler). The 50 bp ladder is recommended in place of the ultra-low range ladder. The tube was wrapped in tin foil (or placed in a dark container) and stored at 4° C.

### Testing

The SeraPure mixture should be tested to ensure that it is working as expected. The test may be conducted using DNA ladder; use Fermentas GeneRuler as NEB ladders may cause problems.

Fresh aliquots of 70% EtOH were prepped. 2 μL GeneRuler were mixed with 18 μL dH₂O. 20 μL GeneRuler mixture was added to a volume of SeraPure and/or AMPure (specific volume depends on whether small fragments are attempting to be excluded). The mixture was incubated 5 minutes at room temperature. It was placed on a magnet stand and the supernatant was removed. 500 μL 70% EtOH was added, the mixture was incubated on the stand for 1 minute, and the supernatant was removed (2×). The beads were placed on a 37° C. heat block for 3-4 min until dry. The beads were rehydrated with 20 μL dH₂O. The tube was placed on a magnet stand, and the supernatant was transferred to a new tube. The supernatant was mixed with 1 μL loading dye and electrophoresed in 1.5% agarose for 60 min at 100 V.

### Example 2

Data from four samples demonstrating enrichment of TCR and BCR transcripts are shown in FIG. 3. Non-enriched samples include single cell transcriptome libraries from cells isolated from a cytobrush sample of a female genital tract, a PBMC sample that was stimulated for 10 days with CEFT-peptide or DMSO alone and a newly thawed PBMC sample using the SeqWell technique. Each library was then iteratively enriched three times for TCRA, TCRB and, in the case of the newly thawed PBMC sample, BCRH using a pool of biotinylated probes. The concentration of TCRA, TCRB, BCRH and the house keeping gene ActB in the pre-enriched and enriched libraries were measured by qPCR. In the left panel of FIG. 3, the ratio of each immune receptor to ActB in each enrichment round is displayed for the cytobrush sample as an example. In the right four panels of FIG. 3, the fold enrichment of the immune receptor transcripts relative to ActB in each enrichment round compared to the pre-enriched library is plotted.

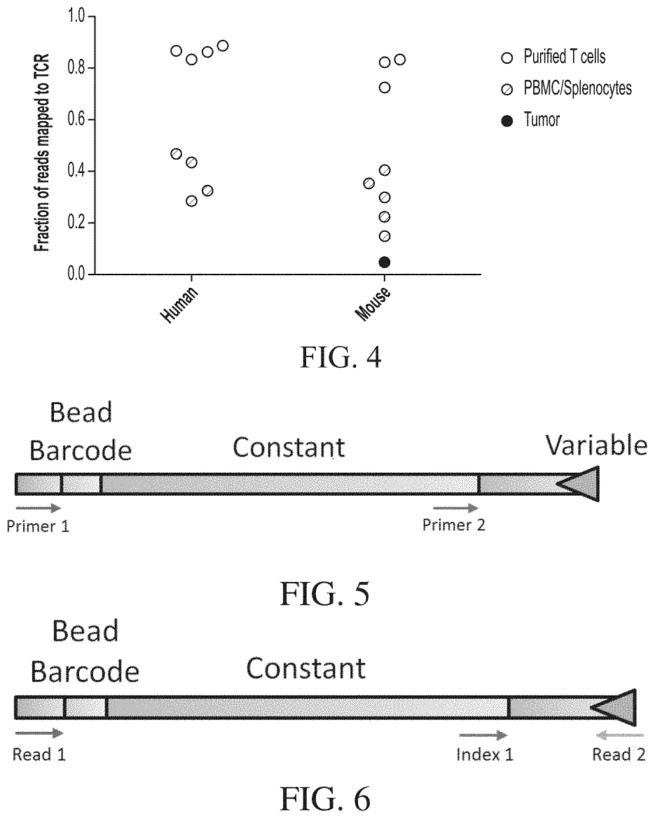

Multiple users have utilized the enrichment technique to sequence TCR transcripts from both human and mouse samples. The fraction of reads mapping to the TCR loci after a single round of enrichment in libraries derived from the different cell populations shown is plotted in FIG. 4. Each dot is a unique sample library. Overall, the fraction of reads that map to the TCR loci correlates with the fraction of cells in the initial sample that were T cells.

### Example 3

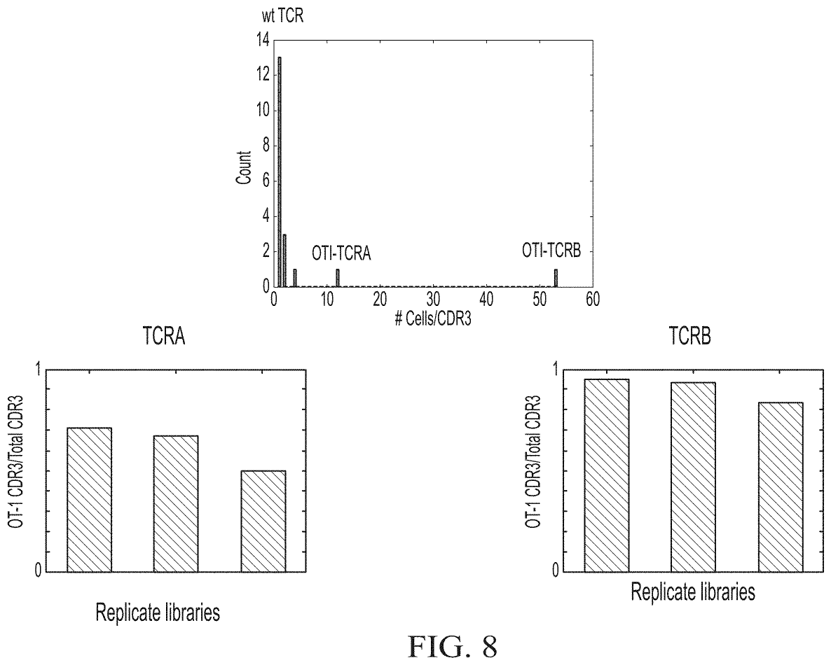

Independent SeqWell libraries created from a mix of T cells isolated from wild type mice and OT-1 Tg mice were enriched and sequenced. In the top panel of FIG. 8, the number of times a unique CDR3 sequence is found associated with a given number of bead barcodes is displayed. Unique T cells are only found associated with a single barcode and are counted in the bar with 1 cell/CDR3. Two CDR3 sequences were found on multiple bead barcodes (12 and 53 cells) and were found to be the OT-I TCRA and TCRB CDR3 sequences respectively. In the bottom two panels of FIG. 8, the ratio of the number of cells found to express the OT-I TCRA or TCRB to the number of unique T cells (1 cell/CDR3) for the three independent libraries is displayed, demonstrating reproducible enrichment.

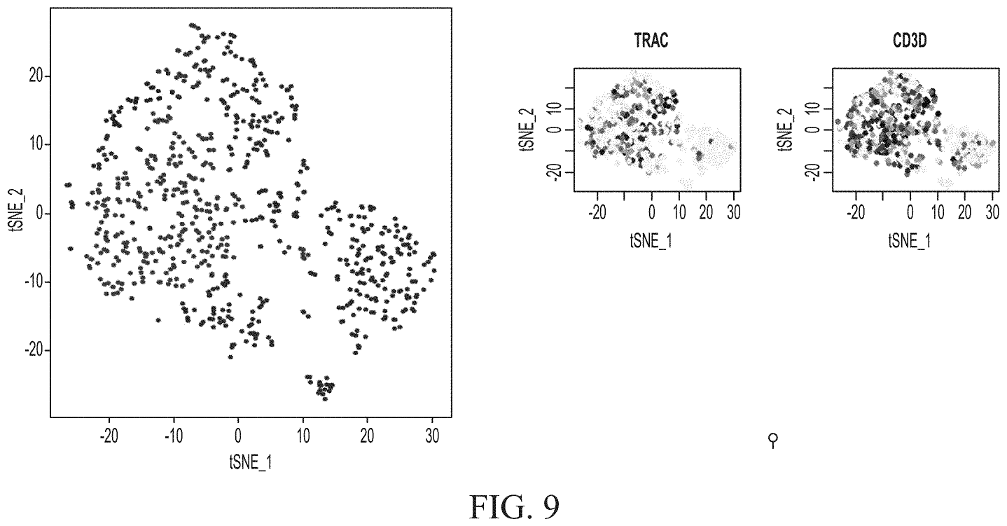

SeqWell-derived single cell transcriptomes created from leukocytes isolated from a mouse lymph node were sequenced. t-distributed stochastic neighbor embedding (rSNE) analysis was used to collapse the data into two dimensions to identify cells with similar transcriptomes, as is shown in the left panel of FIG. 9. Expression levels of TCRA (TRAC) and CD3D were then mapped onto the tSNE plot as a heatmap in order to identify the cell clusters containing T cells, as is shown in the right panel of FIG. 9. 507 cells were defined as T cells.

The same Seqwell library was enriched for TCR transcripts and sequenced. 177 million reads were performed in the total library and 11 million reads were performed in the TCR enriched library. In Table 1 below, the percentage of reads that map to TCR loci before and after enrichment is shown.

TABLE 1
% Total reads mapping to TCR

| | Full library | TCR Enr. Library |
|---|---|---|
| TCRA | 0.22% | 22.1% |
| TCRB | 0.30% | 19.0% |

In Table 2 below, the percentage of the 507 bead barcodes defined as T cells in the previous analysis having at least 20 reads mapping to TCR variable region or defined CDR3 region extracted from the sequencing data are shown.

TABLE 2
% T cell barcodes with mapped TCR data

| | |
|---|---|
| Total library | TCR Enr. Library |

| | >20 Reads | >20 Reads | CDR3 |
|---|---|---|---|
| TCRA | 0.2 | 39 | 23 |
| TCRB | 1.8 | 65 | 46 |
| Both | 0 | 30 | 14 |

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for enriching for T cell receptor- or B cell receptor-encoding transcripts in a library of transcripts comprising:
providing a library of transcripts from a plurality of cells, with transcripts from each cell identified by a unique barcode;
contacting the library of transcripts with a labeled oligonucleotide that is complementary to a constant region of the T cell receptor- or B cell receptor-encoding transcripts; and
separating the labeled oligonucleotide hybridized to the T cell receptor- or B cell receptor-encoding transcripts from the library of transcripts,
thereby obtaining a population of transcripts enriched for T cell receptor- or B cell receptor-encoding transcripts.

2. A method for identifying a T cell receptor- or B cell receptor in a cell comprising:
providing a library of transcripts from a plurality of cells, with each transcript having a 5′ universal primer site followed by a barcode that is unique to each cell;
contacting the library of transcripts with a first sequencing primer that is complementary to the universal primer site and a second sequencing primer that is complementary to a constant region of T cell receptor- or B cell receptor-encoding transcripts;
sequencing from both primers in the same read, such that barcode sequence data for a T cell receptor- or B cell receptor-encoding transcript is generated in conjunction with CDR3 sequence data.

3. A method for producing a library of T cell receptor- or B cell receptor-encoding transcripts, each comprising a same primer sequence at a uniform position in the variable region comprising:
providing:
(i) a library of T cell receptor- or B cell receptor-encoding transcripts;
(ii) a pool of oligonucleotides complementary to all known sequences at a uniform position in the variable region;
(iii) transposase; and
(iv) a transposon comprising the primer sequence.

4. A method for determining an expression profile associated with a T cell or B cell receptor comprising:
providing a library of transcripts from a plurality of cells, with transcripts from each cell identified by a unique barcode;
determining the sequence and the expression level of the transcripts in the library;
selecting the sequence of a T cell receptor or B cell receptor from the library or derived from the library; and
identifying the expression levels of the transcripts having the same barcode as the T cell or B cell receptor;
thereby providing a gene expression profile for the T cell or B cell receptor.
